SEQUENCE LISTING (18 sequences)

SEQ ID NO: 1 - DNA, 36 bp, Artificial Sequence
  Synthetic Construct; CT_F Primer
tgcaacgggttattcactcccnnnnncattgaaact

SEQ ID NO: 2 - DNA, 36 bp, Artificial Sequence
  Synthetic Construct; CT_R Primer
acccataccacaccgctttctnnnnngcctacacgt

SEQ ID NO: 3 - DNA, 24 bp, Artificial Sequence
  Synthetic Construct; CT_DLP(18) Primer
caccttctcgtaccaaagctagaa

SEQ ID NO: 4 - DNA, 37 bp, Artificial Sequence
  Synthetic Construct; NG_F Primer
ctatttttatgagccggaaccgnnnnnaagcgtggga

SEQ ID NO: 5 - DNA, 35 bp, Artificial Sequence
  Synthetic Construct; NG_R Primer
ttgaagttgtcgggaaagccnnnnnttcttgcaac

SEQ ID NO: 6 - DNA, 33 bp, Artificial Sequence
  Synthetic Construct; NG_DLP(18)
ccccgtgcttttccatctttnnnnnttgcgggt

SEQ ID NO: 7 - DNA, 35 bp, Artificial Sequence
  Synthetic Construct; TV_F Primer
cccaaaggctaaccgtgagannnnnatctccctca

SEQ ID NO: 8 - DNA, 33 bp, Artificial Sequence
  Synthetic Construct; TV_R Primer
tcggctgttgtgttgaaagcnnnnncacgctct

SEQ ID NO: 9 - DNA, 23 bp, Artificial Sequence
  Synthetic Construct; TV_DLP(19) Primer
tgatctcacagcctggatggtca

SEQ ID NO: 10 - DNA, 37 bp, Artificial Sequence
  Synthetic Construct; HD_F Primer
aaagaacgtgaaaaagccgaccnnnnnaaaattacta

SEQ ID NO: 11 - DNA, 37 bp, Artificial Sequence
  Synthetic Construct; HD_R Primer
atagcccagaagggttagcaatnnnnngacaatcaat

SEQ ID NO: 12 - DNA, 36 bp, Artificial Sequence
  Synthetic Construct; HD_DLP(20) Primer
cctcggctggtattacgactatcnnnnnagcctagg

SEQ ID NO: 13 - DNA, 31 bp, Artificial Sequence
  Synthetic Construct; HSV1_F Primer
gtcctggtggtgcaaccgnnnnnagttccga

SEQ ID NO: 14 - DNA, 33 bp, Artificial Sequence
  Synthetic Construct; HSV1_R Primer
atacgcacggtcaccccacnnnnntgtgagact

SEQ ID NO: 15 - DNA, 33 bp, Artificial Sequence
  Synthetic Construct; HSV1_DLP(18) Primer
cagccgattacgacgaggatnnnnntgacgagg

SEQ ID NO: 16 - DNA, 32 bp, Artificial Sequence
  Synthetic Construct; HSV2_F Primer
gtcgtctgcgccaaatacgnnnnngcagaccc

SEQ ID NO: 17 - DNA, 32 bp, Artificial Sequence
  Synthetic Construct; HSV2_R Primer
caggcgatggtcaggttgtnnnnntgctttcg

SEQ ID NO: 18 - DNA, 36 bp, Artificial Sequence
  Synthetic Construct; HSV2_DLP(21) Primer
ccgatcactgtgtactacgcagtnnnnnaacgtgcc

(19) United States
(12) Patent Application Publication
(10) Pub. No.: US 2012/0258447 A1
(43) Pub. Date: Oct. 11, 2012

(54) REAL-TIME MULTIPLEXING DETECTION OF TARGET NUCLEIC ACID SEQUENCES WITH ELIMINATION OF FALSE SIGNALS

(75) Inventor: Jong Yoon Chun, Seoul (KR)

(73) Assignee: SEEGENE, INC, SEOUL (KR)

(21) Appl. No.: 13/517,992

(22) PCT Filed: Mar. 15, 2010
(86) PCT No.: PCT/KR2010/001592

§ 371 Date: Jun. 21, 2012

(30) Foreign Application Priority Data
Dec. 24, 2009 (KR) 1-2009-0130604

(51) Int. Cl. G01N 21/64; C12Q 1/68

(52) U.S. Cl. 435/5; 435/6.12

(57) ABSTRACT

The present invention relates to the real-time multiplex detection of at least three target nucleic acid sequences with elimination of false positive signals. Unlikely to conventional real-time multiplex PCR methods, the present invention comprises two different amplification reactions in different reaction vessels from each other: a primary multiplex PCR for obtaining amplicons and a secondary nested real-time multiplex PCR using the amplicons. The present invention permits to eliminate the false positive signals generated by the dimer formation of labeled primers, false positive signals

### BACKGROUND OF THE INVENTION

1. Field of the Invention

The present invention relates to the detection of a target nucleic acid sequence with elimination of false signals.

2. Description of the Related Art

A target nucleic acid amplification process is prevalently involved in most of technologies for detecting target nucleic acid sequences. Nucleic acid amplification is a pivotal process for a wide variety of methods in molecular biology, such that various amplification methods have been proposed. For example, Miller, H. I. et al. (WO 89/06700) amplified a nucleic acid sequence based on the hybridization of a promoter/primer sequence to a target single-stranded DNA (“ssDNA”) followed by transcription of many RNA copies of the sequence.

The most predominant process for nucleic acid amplification known as polymerase chain reaction (hereinafter referred to as “PCR”) is based on repeated cycles of denaturation of double-stranded DNA, followed by oligonucleotide primer annealing to the DNA template, and primer extension by a DNA polymerase (Mullis et al. U.S. Pat. Nos. 4,683,195, 4,683,202, and 4,800,159; Saiki et al., (1985) Science 230, 1350-1354).

PCR-based techniques have been widely used not only for amplification of a target DNA sequence, but also for scientific applications or methods in the fields of biological and medical research, such as reverse transcriptase PCR (RT-PCR), differential display PCR (DD-PCR), cloning of known or unknown genes by PCR, rapid amplification of cDNA ends (RACE), arbitrary priming PCR (AP-PCR), multiplex PCR, SNP genome typing, and PCR-based genomic analysis (McPherson and Moller, (2000) PCR. BIOS Scientific Publishers, Springer-Verlag New York Berlin Heidelberg, N.Y.).

In the meantime, a variety of real-time PCR procedures have been proposed for detecting target nucleic acids based on nucleic acid amplification. The real-time PCR methods have been highlighted in the senses that they can detect amplicons of target sequences in a real-time manner or more accurate quantitative manner and be free from contaminations. The real-time PCR procedures generally use labeled oligonucleotides or DNA-binding dyes to generate signals indicative of target sequences during amplification reactions.

As representative of DNA-binding dyes, SYBR Green intercalates between DNA double strands to exhibit fluorescence. However, since SYBR Green is very likely to non-specifically intercalate between the DNA strands, it is not suitable in specific target detection. To make matters worse, the SYBR Green technology has to identify amplified products by melting curve analysis when it is applied to real-time multiplex detection and therefore is considered unsuitable in terms of analysis time and efficiency.

Most of real-time PCR methods employ labeled oligonucleotides. The conventional real-time PCR methods have their prominent features in terms of (i) uses of labeled oligonucleotides (primers or probes), (ii) formation of unique conformation or structure of labeled oligonucleotides (e.g., hairpin-loop structure), (iii) the number of labels linked to oligonucleotides, (iv) principles underlying signal generation, or (v) characteristics of nucleic acid polymerases used. The illustrative examples include TaqMan™ probe method (U.S. Pat. No. 5,210,015), Molecular Beacon method (Tyagi et al, Nature Biotechnology v.14 MARCH 1996), Scorpion method (Whitcombe et al, Nature Biotechnology 17:804-807 (1999)), Sunrise (or Amplifluor) method (Nazarenko et al, Nucleic Acids Research, 25(12):2516-2521 (1997), and U.S. Pat. No. 6,117,635), Lux method (U.S. Pat. No. 7,537,886), CPT (Duck P, et al., Biotechniques, 9:142-148 (1990)), LNA method (U.S. Pat. No. 6,977,295), and Plexor method (Sherrill C B, et al., Journal of the American Chemical Society, 126:4550-45569 (2004)).

Together with developments of real-time PCR procedures, real-time multiplex PCR methods for simultaneously detecting multiple target sequences have been being suggested. The real-time multiplex PCR methods have some plausible advantages in light of: time-effectiveness, cost-effectiveness, convenience and highthrough-put performance (R. N. Gunson, et al., Journal of Clinical Virology, 43:372-375 (2008)).

Notwithstanding this, primers or probes in the conventional real-time multiplex PCR methods are very likely to generate non-specific hybridization (R. N. Gunson, et al., Journal of Clinical Virology, 43:372-375 (2008)). In the case of labeled primer-based real-time multiplex PCR, the dimer formation of labeled primers is considered as a main hurdle because it results in occurrence of false positive signals.

The real-time multiplex PCR method will become the most promising technology for simultaneously detecting multiple target sequences only where the false positive signals associated with performance of real-time multiplex PCR procedures are substantially or completely eliminated.

Throughout this application, various patents and publications are referenced, and citations are provided in parentheses. The disclosure of these patents and publications in their entities are hereby incorporated by references into this application in order to more fully describe this invention and the state of the art to which this invention pertains.

### SUMMARY OF THE INVENTION

The present inventor has made intensive efforts to develop a novel approach for a real-time multiplex detection of multiple target nucleic acid sequences with completely eliminating false positive signals generated by the dimer formation of labeled primers during conventional labeled primer-based real-time multiplex PCR (polymerase chain reaction) methods. As results, the present inventor has finally proposed a promising method for detection of at least three target nucleic acid sequences performed in such a manner that at least three target nucleic acid sequences are first multiple-amplified to give amplicons and the amplicons are in turn amplified by a nested real-time multiplex PCR using labeled nested primers. The present inventor has verified that the novel method permits to detect multiple target nucleic acid sequences in a real-time multiplex manner with completely eliminating false positive signals caused by the dimer formation of the labeled primers and also with enhancing sensitivity and specificity.

Accordingly, it is an object of this invention to provide a method of a real-time multiplex detection of at least three types of target nucleic acid sequences with elimination of false signals in a real-time multiplex PCR.

It is another object of this invention to provide a kit for a real-time multiplex PCR detection of at least three types of target nucleic acid sequences with elimination of false signals in a real-time multiplex PCR.

Other objects and advantages of the present invention will become apparent from the detailed description to follow, taken in conjugation with the appended claims and drawings.

### BRIEF DESCRIPTION OF THE DRAWINGS

FIG. 1 schematically represents the present method involving a primary multiplex PCR and a secondary nested real-time multiplex PCR. Panel A represents the primary multiplex PCR. The forward and reverse primers are hybridized with target nucleic acid sequences and extended. By repetition of PCR cycles including denaturation, annealing and extension, multiple target nucleic acid sequences are amplified. The primers for the primary multiplex PCR may have any form or structure such as DPO (dual priming oligonucleotide) structure and conventional structures known to one of skill in the art. Panel B represents the secondary nested real-time multiplex PCR using labeled nested primers. At least one of the forward and reverse primers having at least one label is a nested primer hybridizable with the internal sequences of the corresponding amplicons obtained from the primary multiplex PCR. The representatives of primers capable of being used in the secondary nested real-time multiplex PCR include TSG primer, THD primer, Scorpion, Sunrise, Lux, Plexor, and Antiprimer having a single label or dual label system. The secondary nested real-time multiplex PCR using the labeled nested primers can provide satisfactory signals indicating the presence of target nucleic acid sequences by performing reaction cycles to the extent that the target signals are generated but the false positive signals not generated, which allows for elimination of false positive signals generated by the dimer formation of labeled primers. Furthermore, the present method leads to enhancement in PCR target specificity and sensitivity.

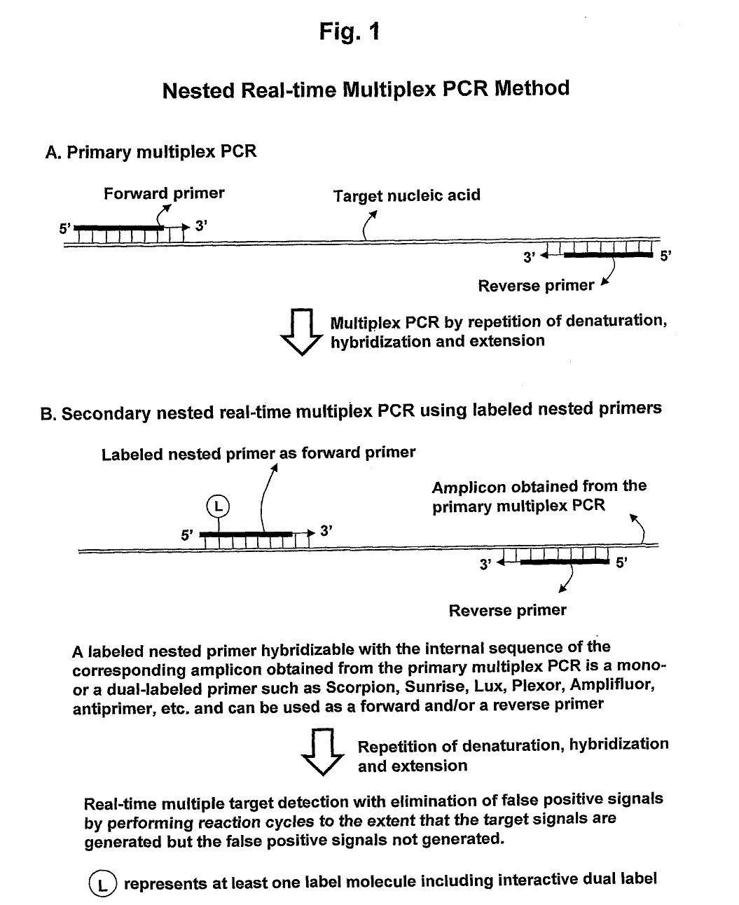

FIG. 2 represents the results of the conventional real-time multiplex PCR method performed with the three primer pairs for the detection of C. trachomatis, N. gonorrhoeae and T. vaginalis. The conventional method gave rise to the false positive signals caused by the dimer formation of the labeled primers.

FIG. 3 represents the results of the nested real-time multiplex PCR method of the present invention performed with the three primer pairs for the detection of C. trachomatis, N. gonorrhoeae and T. vaginalis. The results showed that the true signal indicative of the presence of the target nucleic acid was obtained with elimination of false positive signals by the secondary nested real-time multiplex PCR carried out for 30 cycles.

FIG. 4 represents the results of the conventional real-time multiplex PCR method performed with the three primer pairs for the detection of H. ducreyi, Herpes simplex virus 1 and Herpes simplex virus 2. The conventional method gave rise to the false positive signals caused by the dimer formation of the labeled primers.

FIG. 5 represents the results of the nested real-time multiplex PCR method performed with the three primer pairs for the detection of H. ducreyi, Herpes simplex virus 1 and Herpes simplex virus 2. The results showed that the true signal indicative of the presence of the target nucleic acid was obtained with elimination of false positive signals by the secondary nested real-time multiplex PCR carried out for 30 cycles.

FIG. 6 represents the results to show the sensitivity and specificity of the nested real-time multiplex PCR method of the present invention. The secondary nested real-time multiplex PCR carried out for 30 cycles was conducted with the three primer pairs for the detection of H. ducreyi, Herpes simplex virus 1 and Herpes simplex virus 2. The genomic DNA of H. ducreyi was used as a template with 10-fold serial dilutions (from 1000 fg to 1 fg).

FIG. 7 represents the results to show the sensitivity and specificity of the nested real-time multiplex PCR method of the present invention. The secondary nested real-time multiplex PCR carried out for 30 cycles was conducted with the three primer pairs for the detection of H. ducreyi, Herpes simplex virus 1 and Herpes simplex virus 2. The genomic DNA of Herpes simplex virus 1 was used as a template with 10-fold serial dilutions (from 1000 fg to 1 fg).

FIG. 8 represents the results of the nested real-time multiplex PCR method of the present invention for the detection of H. ducreyi, Herpes simplex virus 1 and Herpes simplex virus 2. The genomic DNAs of H. ducreyi, Herpes simplex virus 1 and Herpes simplex virus 2 were used as templates.

### DETAILED DESCRIPTION OF THIS INVENTION

In one aspect of the present invention, there is provided a method of a real-time multiplexing detection of at least three types of target nucleic acid sequences in a sample with elimination of false signals in a real-time multiplex PCR (polymerase chain reaction), which comprises the steps of:

(a) performing a primary multiplex PCR for amplifying the target nucleic acid sequences in a reaction vessel, wherein the primary multiplex PCR is carried out using at least three primer pairs and a template-dependent nucleic acid polymerase under conditions sufficient to amplify the at least three types of target nucleic acid sequences in order to obtain the amplicons of the target nucleic acid sequences in the sample;

(b) preparing a secondary nested real-time multiplex PCR reaction mixture in a reaction vessel distinctly different from the reaction vessel used in the step (a), wherein the secondary nested real-time multiplex PCR reaction mixture contains (i) the amplicons obtained in the step (a), (ii) at least three primer pairs for secondary the nested real-time multiplex PCR of the amplicons in which at least one primer of each pair of the at least three primer pairs is a nested primer hybridizable with an internal sequence of the corresponding amplicon and (iii) a template-dependent nucleic acid polymerase, wherein each pair of the at least three primer pairs comprises at least one nested primer having a label generating the signal indicative of the presence of the target nucleic acid sequence during the secondary nested real-time multiplex PCR;

(c) performing the secondary nested real-time multiplex PCR using the reaction mixture of the step (b) by carrying out at least two cycles of primer annealing, primer extension and denaturation, wherein the signal indicative of the presence of the target nucleic acid sequence is generated from each of the labeled primers during the cycles, such that the secondary nested real-time multiplex PCR can be carried out for the number of cycles not to generate the false signals but to generate the signals indicative of the presence of the target nucleic acid sequences; and

(d) detecting the signal indicative of the presence of the target nucleic acid sequence, wherein the detection is performed for each cycle of the repetition of step (c), at the end of the repetition of step (c) or at each of predetermined time intervals during the repetition of step (c), whereby the signals indicative of the presence of the target nucleic acid sequences are obtained in a real-time manner without the false signals.

Generally, the conventional real-time PCR technologies using labeled probes or primers have serious problems when it is conducted for the simultaneous detection of multiple target sequences. For instance, those using labeled probes need many oligonucleotides such as primers and probes for amplification and detection, respectively. In this regard, it is very difficult to determine the proper sequences of oligonucleotides as primers and probes and also difficult to optimize reaction conditions of real-time multiplex PCR. In addition, they become more likely to generate false positive signals generated by the dimer formation or non-specific hybridization of the oligonucleotides. Therefore, the probe-based real-time PCR methods are considered to be unsuitable in the simultaneous detection of multiple target sequences.

For the conventional real-time PCR approaches using a labeled primer, there is also an unavoidable shortcoming associated with the dimer formation of the labeled primer which causes false positive signal generation in the real-time detection of a target sequence. In particular, such a problem becomes more serious in real-time multiplex PCR for the simultaneous detection of multiple target sequences. Since the real-time multiplex PCR requires multiple labeled primers, false positive signals are likely to be generated by the dimer formation of the labeled primers.

Furthermore, where target nucleic acid sequences are present in a very low amount or not present, it is significantly difficult to discriminate or determine whether the signals obtained from the labeled primer-based real-time multiplex PCR are true by the presence of the target nucleic acid sequences or false by the dimer formation of the labeled primers.

In the meantime, the sequence selection of labeled primers is generally used as a strategy for avoiding the dimer formation problem. However, this strategy has still a limitation in completely eliminating the dimer problem.

The present inventor has made intensive efforts to develop a novel approach for a real-time multiplex detection of multiple target nucleic acid sequences with completely eliminating false positive signals generated by the dimer formation of labeled primers during conventional labeled primer-based real-time multiplex PCR methods. As results, the present inventor has finally proposed a promising method for detection of at least three target nucleic acid sequences performed in such a manner that at least three target nucleic acid sequences are first multiple-amplified to give amplicons and the amplicons are in turn amplified by a nested real-time multiplex PCR using labeled nested primers. The present inventor has verified that the novel method permits to detect multiple target nucleic acid sequences in a real-time multiplex manner with completely eliminating false positive signals generated by the dimer formation of the labeled primers.

To my best knowledge, it is first proposed by the present inventor that at least three target nucleic acid sequences are able to be simultaneously detected using at least three labeled nested primers in a nested real-time manner with eliminating or discriminating false positive signals generated by the dimer formation of the labeled primers.

The elimination (or removal) of false positive signals generated by the dimer formation of labeled primer is ascribed to intricate combination of the following three strategies. The first strategy is a pre-amplification of multiple target nucleic acid sequences that must be performed separately from a real-time signal generating process. The second one is to utilize labeled nested primers hybridizable with internal sequences of the corresponding amplicons produced by the pre-amplification. The third one is to restrict a cycle number of real-time multiplex PCR reaction to the extent that the target signals are generated but the false positive signals generated by the dimer formation not generated.

In the present invention, the target nucleic acid sequences are first pre-amplified by a primary multiplex PCR and then the amplicons are used as templates for a secondary nested real-time multiplex PCR. Since the pre-amplification increases the amount of the target nucleic acid sequences, threshold cycle (Ct) values of signals for target nucleic acid sequences during the secondary nested real-time multiplex PCR become much lower than those Ct values obtained without pre-amplification. In contrast, the Ct values of the false positive signals generated by the dimer formation of labeled primers are not changed regardless of the use of pre-amplification. Therefore, this phenomenon allows that the signals indicative of the presence of target nucleic acid sequences are generated before the generation of the false positive signals, and eventually the true target signals are discriminative from the false positive signals.

When the primers for the primary multiplex PCR are used as the labeled primers for the secondary real-time multiplex PCR, false positive signals are likely to be generated by the interaction of the labeled primers with non specific products or dimer products which are produced during the primary multiplex PCR. In contrast, when the labeled primers for the secondary real-time multiplex PCR are nested primers hybridizable with internal sequences of the corresponding amplicons produced from the primary multiplex PCR, such false positive signals are fundamentally prevented by avoiding the interaction of the labeled nested primers with non specific products or dimer products of the primary multiplex PCR.

Finally, a cycle number in the secondary nested real-time multiplex PCR of the present invention is restricted to the extent that the multiple target signals are generated but the false positive signals by the dimer formation not generated. Therefore, the restriction of the cycle number allows for elimination of the false positive signals generated by the dimer formation, and eventually the detection of true signals indicating the presence of target nucleic acid sequences.

Furthermore, the present inventor has found that false positive signals, e.g., resulting from the dimer formation of labeled primers in conventional real-time multiplex PCR are very likely to be produced after 30 cycles. Therefore, the present inventor has elucidated that the production of false positive signals generated by the dimer formation is prevented by performing the secondary nested real-time multiplex PCR for no more than 30 cycles. Since the secondary nested real-time multiplex PCR of the present invention are performed using pre-amplified target nucleic acid sequences, the present inventor has found that the signals indicating the presence of target nucleic acid sequences are obtained within 30 cycles.

In accordance with the present invention, the secondary nested real-time multiplex PCR is performed using at least three primer pairs in which at least one primer of each pair of the at least three primer pairs is a nested primer hybridizable with the internal sequence of the corresponding amplicon obtained from the primary multiplex amplification, giving rise to occurrence of nested PCR effects such as enhanced specificity and sensitivity. The secondary nested real-time multiplex PCR produces the effects similar to those of the second round PCR in a conventional nested PCR. In other words, the primary multiplex PCR of the present invention corresponds to the first round PCR in a conventional nested PCR and the secondary nested real-time multiplex PCR of the present invention to the second round PCR in a conventional nested PCR.

Especially, one of the features of the present method assigns labeled primers to nested primers in the secondary nested real-time multiplex PCR. Therefore, the present invention ensures a dramatic elevation of target specificity in the detection of multiple target nucleic acid sequences by nested amplification effects in the real-time PCR, resulting in elimination or reduction of false positive signals owing to non-specific hybridization with non-target sequences in samples.

Furthermore, the nested effects of the present method dramatically increase sensitivity comparing to those of the conventional real-time multiplex PCR methods. In other words, when the templates of multiple target sequences are present in a very low amount, conventional real-time multiplex PCR methods may be unable to detect target signals due to the limit of detection. In contrast, the present method allows the accurate detection of target sequences present in a very low amount by dramatically increased sensitivity with help of nested amplification effects.

In this connection, the present invention is called “Nested real-time Multiple PCR Method by a Sequential Combination of Primary Multiplex PCR and Secondary Nested Real-time Multiplex PCR”.

The term “Primary Multiplex PCR” used for expressing the features of the present invention means a multiplex PCR reaction performed using at least three primer pairs in order to obtain the amplicons of target nucleic acid sequences in a sample.

The term “Secondary Nested Real-time Multiplex PCR” used for expressing the features of the present invention means a real-time multiplex PCR reaction performed using the amplicons obtained from the primary multiplex PCR as templates and at least three primer pairs of which at least one of each pair is a labeled nested primer hybridizable with an internal sequence of the corresponding amplicons, generating signals indicating the presence of target nucleic acid sequences in a real-time manner.

The term used herein “elimination (or removal) of false positive signals” or “elimination (or removal) of false positive data” refers to decrease in false positive signals with comparing to conventional real-time multiplex PCR reactions which do not have pre-amplification of target nucleic acid sequences. Preferably, the term refers to substantial elimination of false positive signals, more preferably complete elimination of false positive signals.

The samples used in the present invention include any samples. Preferably, biosamples are analyzed by the present method. The biosamples of plant, animal, human, fungus, bacterium and virus origin can be analyzed. If a sample of a mammal or human origin is analyzed, the sample can be derived from a particular tissue or organ. Exemplary tissues include connective, epithelium, muscle or nerve tissue. Exemplary organs include eye, brain, lung, liver, spleen, bone marrow, thymus, heart, lymph, blood, bone, cartilage, pancreas, kidney, gall bladder, stomach, intestine, testis, ovary, uterus, rectum, nervous system, gland and internal blood vessels. The biosample to be analyzed may include any cell, tissue, or fluid from a biological source, or any other medium that can advantageously be analyzed according to this invention, including a sample drawn from human, a sample drawn from an animal, a sample drawn from food designed for human or animal consumption. Also, the biosample to be analyzed includes a body fluid sample including blood, serum, plasma, lymph, milk, urine, faeces, ocular fluid, saliva, semen, brain extracts (e.g., brain homogenates), spinal cord fluid (SCF), appendix, spleen and tonsillar tissue extracts.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. The primer is preferably single stranded for maximum efficiency in amplification. Preferably, the primer is an oligodeoxyribonucleotide. The primer of this invention may be comprised of naturally occurring dNMP (i.e., dAMP, dGM, dCMP and dTMP), modified nucleotide, universal nucleotide or non-natural nucleotide. The primer may also include ribonucleotides.

The primer must be sufficiently long to prime the synthesis of extension products in the presence of the agent for polymerization. The exact length of the primers will depend on many factors, including temperature, application, and source of primer. The term “annealing” or “priming” as used herein refers to the apposition of an oligodeoxynucleotide or nucleic acid to a template nucleic acid, whereby the apposition enables the polymerase to polymerize nucleotides into a nucleic acid molecule which is complementary to the template nucleic acid or a portion thereof.

The term “hybridizing” used herein refers to the formation of a double-stranded nucleic acid from complementary single stranded nucleic acids. The hybridization may occur between two nucleic acid strands perfectly matched or substantially matched with some mismatches. The complementarity for hybridization may depend on hybridization conditions, particularly temperature. There is no intended distinction between the terms “annealing” and “hybridizing”, and these terms will be used interchangeably.

The term “multiplex detection or multiplexing detection” used herein means a simultaneous detection of multiple target nucleic acid sequences in a reaction vessel (e.g., a reaction tube).

The term used herein “target nucleic acid”, “target nucleic acid sequence” or “target sequence” refers to a nucleic acid sequence of interest for detection, which is annealed to or hybridized with a primer or probe under hybridization, annealing or amplifying conditions.

The term used herein “multiplex PCR” means the simultaneous amplification of multiplex targets by polymerase chain reaction in a reaction vessel.

According to the present invention, a multiplex PCR is performed with at least three primer pairs capable of amplifying at least three target nucleic acid sequences in a reaction vessel, thereby obtaining the amplicons of the target nucleic acid sequences in a sample.

The multiplex PCR of target nucleic acid sequences may be carried out in accordance with PCR methods known in the art. Preferably, the multiplex PCR reactions are carried out according to the process disclosed in U.S. Pat. Nos. 4,683,195, 4,683,202, and 4,800,159.

According to a preferred embodiment, the primary multiplex PCR of the present invention is performed for no more than 50 cycles, more preferably no more than 40 cycles, still more preferably no more than 30 cycles, still much more preferably no more than 20 cycles.

According to a preferred embodiment, the primary multiplex PCR of the present invention is performed for at least 2 cycles, more preferably at least 5 cycles, still more preferably at least 10 cycles, still much more preferably at least 15 cycles.

According to a preferred embodiment, the number of cycles of the primary multiplex PCR is determined to provide amplicons whose amounts permit to generate signals indicating the presence of target nucleic acid sequences during the secondary nested real-time multiplex PCR.

The target nucleic acid sequences in a sample may be either DNA or RNA. The molecule may be in either a double-stranded or single-stranded form. Where the nucleic acid as starting material is double-stranded, it is preferred to render the two strands into a single-stranded or partially single-stranded form. Methods known to separate strands includes, but not limited to, heating, alkali, formamide, urea and glycoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, a random hexamer or an oligonucleotide dT primer hybridizable to poly A tail of mRNA is used. The oligonucleotide dT primer is comprised of dTMPs, one or more of which may be replaced with other dNMPs so long as the dT primer can serve as primer. Reverse transcription can be done with reverse transcriptase that has RNase H activity. If one uses an enzyme having RNase H activity, it may be possible to omit a separate RNase H digestion step by carefully choosing the reaction conditions.

The primers used in the present invention are hybridized or annealed to sites on target nucleic acid sequences (as templates) that double-stranded structure is formed. Conditions of nucleic acid annealing suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the primary multiplex PCR step of the present method, which includes “Klenow” fragment of E. coli DNA polymerase I, a thermostable DNA polymerase, and bacteriophage T7 DNA polymerase. Preferably, the polymerase is a thermostable DNA polymerase which may be obtained from a variety of bacterial species, including Thermus aquaticus (Taq), Thermus thermophilus (Tth), Thermus filiformis, Thermis flavus, Thermococcus literalis, and Pyrococcus furiosus (Pfu).

When a polymerization reaction is being conducted, it is preferable to provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

All of the enzymes used in the primary multiplex PCR or the secondary nested real-time multiplex PCR may be active under the same reaction conditions. Indeed, buffers exist in which all enzymes are near their optimal reaction conditions. Therefore, the multiplex PCR processes of the present invention can be done in a single reaction volume without any change of conditions such as addition of reactants.

The annealing or hybridization for the multiplex PCR in the present method is performed under stringent conditions that allow for specific binding between a nucleotide sequence and primers. Such stringent conditions for annealing will be sequence-dependent and varied depending on environmental parameters. Preferably, the annealing temperature ranges from 40° C. to 75° C., more preferably from 45° C. to 68° C., most preferably from 50° C. to 65° C.

According to a preferred embodiment, at least one primer of the at least three primer pairs for the primary multiplex PCR or the secondary nested real-time multiplex PCR has a dual priming oligonucleotide (DPO) structure represented by the following general formula I:

5′-Xₚ—Y_{q}—Zᵣ-3′ (I)

wherein, Xₚ represents a 5′-first priming portion having a hybridizing nucleotide sequence complementary to the target nucleic acid; Y_{q} represents a separation portion comprising at least three universal bases, Zᵣ represents a 3′-second priming portion having a hybridizing nucleotide sequence complementary to the target nucleic acid; p, q and r represent the number of nucleotides, and X, Y, and Z are deoxyribonucleotides or ribonucleotides; the Tₘ of the 5′-first priming portion is higher than that of the 3′-second priming portion and the separation portion has the lowest Tₘ in the three portions; the separation portion separates the 5′-first priming portion from the 3′-second priming portion in terms of annealing events to the target nucleic acid, whereby the annealing specificity of the oligonucleotide are determined dually by the 5′-first priming portion and the 3′-second priming portion such that the overall annealing specificity of the primer is enhanced;

The DPO structure as a primer version of DSO (dual specificity oligonucleotide) was first proposed by the present inventor (see WO 2006/095981; Chun et al., Dual priming oligonucleotide system for the multiplex detection of respiratory viruses and SNP genotyping of CYP2C19 gene, Nucleic Acid Research, 35:6e40 (2007)). The DPO embodies a novel concept in which its hybridization or annealing is dually determined by the 5′-high Tₘ specificity portion (or the 5′-first priming portion) and the 3′-low Tₘ specificity portion (or the 3′-second priming portion) separated by the separation portion, exhibiting dramatically enhanced hybridization specificity (see WO 2006/095981; Kim et al., Direct detection of lamivudine-resistant hepatitis B virus mutants by multiplex PCR using dual-priming oligonucleotide primers, Journal of Virological Methods, 149:76-84 (2008); Kim, et. al., Rapid detection and identification of 12 respiratory viruses using a dual priming oligonucleotide system-based multiplex PCR assay, Journal of Virological Methods, doi:10.1016/j.jviromet.2008.11.007 (2008); Horii et. al., Use of dual priming oligonucleotide system to detect multiplex sexually transmitted pathogens in clinical specimens, Letters in Applied Microbiology, doi:10.111/j.1472-765X2009.02618x(2009)). As such, the DPO has eventually two primer segments with distinct hybridization properties: the 5′-first priming portion that initiates stable hybridization, and the 3′-second priming portion that mainly determines target specificity.

The primers having the DPO structure are responsible in part for successful multiplex amplification reactions with eliminated false positive signals.

According to a preferred embodiment, the universal base in the separation portion is selected from the group consisting of deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof. More preferably, the universal base is deoxyinosine, 1-(2′-deoxy-beta-D-ribofuranosyl)-3-nitropyrrole or 5-nitroindole, most preferably, deoxyinosine.

Preferably, the separation portion comprises contiguous nucleotides having at least three, more preferably at least four, most preferably at least five universal bases. Alternatively, the separation portion comprises 3-10, 3-7 or 5-7 contiguous nucleotides.

Preferably, in the DPO structure the 5′-first priming portion is longer than the 3′-second priming portion. The 5′-first priming portion is preferably 15-60 nucleotides, more preferably 15-40 nucleotides, still more preferably 15-25 nucleotides in length. It is preferable that the 3′-second priming portion is 3-15 nucleotides, more preferably 5-15 nucleotides, still more preferably 6-13 nucleotides in length. The separation portion is preferably 3-10 nucleotides, more preferably 4-8 nucleotides, most preferably 5-7 nucleotides in length. According to a preferred embodiment, the Tₘ of the 5′-first priming portion ranges from 40° C. to 80° C., more preferably 45° C. to 65° C. The Tₘ of the 3′-second priming portion ranges preferably from 10° C. to 40° C. It is preferable that the Tₘ of the separation portion ranges from 3° C. to 15° C.

In the step (a) for the primary multiplex PCR, at least three primer pairs, preferably at least four primer pairs and more preferably at least five primer pairs are used.

Following the primary multiplex PCR of target nucleic acid sequences, the secondary nested real-time multiplex PCR is carried out in a reaction vessel distinctly different from the reaction vessel in the step (a) for the primary multiplex PCR. The secondary nested real-time multiplex PCR is performed using at least three primer pairs of which at least one primer of each pair is a nested primer hybridizable with an internal sequence of each of the corresponding amplicons obtained from the step (a). By the secondary nested real-time multiplex PCR, the at least three types of target nucleic acid sequences are capable of being detected in a real-time fashion.

According to the preferable embodiment of the present invention, the forward and reverse primers of at least one primer pair for the secondary nested multiplex PCR are nested primers hybridizable with an internal sequence of the corresponding amplicon obtained from the step (a).

The term used herein “nested primer” means the primer hybridizable with an internal sequence of the corresponding amplicon obtained from the step (a). In other words, the nested primer is derived from nucleotide sequences within the first targeted nucleotide sequence and flanks a second, smaller targeted nucleotide sequence contained within the first targeted nucleotide sequence.

The term used herein “an internal sequence of amplicon” means a sequence of amplicon spanning a sequence positioned inward by at least one nucleotide.

Where the step (c) for the secondary nested real-time multiplex PCR is carried out, each pair of the at least three primer pairs comprises at least one nested primer having a label generating a signal indicative of the presence of the target nucleic acid sequence during the secondary nested real-time multiplex PCR.

According to a preferred embodiment, when the primer used in the secondary nested real-time multiplex PCR has the label generating the signal indicative of the presence of the target nucleic acid sequence, it is a nested primer hybridizable with an internal sequence of the corresponding amplicon.

In the present invention, the signals indicating the presence of target nucleic acid sequences are generated without false positive signals generated by the dimer formation of labeled nested primers in the secondary nested real-time multiplex PCR because it uses pre-amplified products.

According to a preferred embodiment, the signals indicating the presence of target nucleic acid sequences are generated within 30 cycles, more preferably 25 cycles, still more preferably 20 cycles in the secondary nested real-time multiplex PCR.

In the present invention, the secondary nested real-time multiplex PCR of the step (c) is carried out for the number of cycles not to generate the false signals but to generate the signals indicative of the target nucleic acid sequences

According to a preferred embodiment, the secondary nested real-time multiplex PCR of the step (c) is carried out for no more than 30 cycles, more preferably 25 cycles, still more preferably no more than 20 cycles. The secondary nested real-time multiplex PCR is generally carried out for at least 2 cycles, preferably at least 3 cycles, more preferably at least 5 cycles.

In accordance with the present invention, the number of cycles (or Ct value) to generate false positive signals owing to the dimer formation of labeled nested primers may be determined in the absence of template using labeled nested primers used in the secondary nested real-time multiplex PCR.

According to one embodiment of the present invention, the products of the primary multiplex amplification PCR are able to be aliquoted into at least one separate vessel and each vessel is used for the detection of at least three different types of target nucleic acids in the secondary nested multiplex PCR. For example, where the primary 10-plex PCR is carried out to amplify ten different target nucleic acid sequences with 10 primer pairs, the amplified products are aliquoted into three different vessels and then each vessel are used for subtyping, grouping or identification of at least three target nucleic acid sequences in a real-time manner.

According to a preferred embodiment, the labels on at least three primer pairs are different from each other or the same.

According to a more preferred embodiment, the label of each pair of at least three primer pairs is different from each other or the same.

The expression used herein “the labels on primers are different” refers to (i) difference in terms of number, type, absorption or emission wavelength of labels linked to primers; or (ii) difference in signal generating mechanism of labels linked to primers (e.g., signal generation by enzymatic cleavage, three-dimensional conformation change of primers having labels or incorporation of labeled primers into double-stranded PCR products).

The expression used herein “the labels on primers are the same” refers to (i) the sameness in terms of number, type, absorption or emission wavelength of labels linked to primers; and (ii) the sameness in signal generating mechanism of labels linked to primers (e.g., signal generation by enzymatic cleavage, three-dimensional conformation change of primers having labels or incorporation of labeled primers into double-stranded PCR products).

According to a preferred embodiment, the term “a label” is used to mean at least one label.

According to a preferred embodiment, the labeled primer comprises a single label or an interactive dual label.

Preferably, the label is positioned on a complementary site to the target nucleic acid sequence.

According to a preferred embodiment, the labeled primer has a linear structure or intermolecular structure prior to hybridization with target nucleic acid sequences. The examples of the intermolecular structure are a hairpin-loop or G-quartet structure.

The label useful in this invention includes any label known to one of skill in the art. Most of labels are composed of a single molecule or a single atom label; however some labels (e.g., interactive label system) composed of at least two or more label molecules or atoms. According to a preferred embodiment, the label linked to primers is a chemical label, an enzymatic label, a radioactive label, a fluorescent label, a luminescent label, a chemiluminescent label or a metal label (e.g., gold).

According to a preferred embodiment, the label is a fluorescent label, preferably mono fluorescent label, and more preferably an interactive label system, still more preferably FRET label system.

The interactive label system is a signal generating system in which energy is passed non-radioactively between a donor molecule and an acceptor molecule. As a representative of the interactive label system, the FRET (fluorescence resonance energy transfer) label system includes a fluorescent reporter molecule (donor molecule) and a quencher molecule (acceptor molecule). In FRET, the energy donor is fluorescent, but the energy acceptor may be fluorescent or non-fluorescent.

In another form of interactive label systems, the energy donor is non-fluorescent, e.g., a chromophore, and the energy acceptor is fluorescent. In yet another form of interactive label systems, the energy donor is luminescent, e.g. bioluminescent, chemiluminescent, electrochemiluminescent, and the acceptor is fluorescent.

More preferably, the signal indicative of the target nucleic acid sequence is generated by interactive label systems, most preferably the FRET label system.

Where the FRET label is used, the locations of a fluorescent reporter molecule and a quencher molecule may be varied so long as the quencher molecule quenches the fluorescence of the reporter molecule. For example, the fluorescent reporter molecule and the quencher molecule may be separated by 5-50, preferably 5-40 and more preferably 5-30 nucleotides.

According to a preferred embodiment, the fluorescent reporter molecule or the quencher molecule on the labeled primer is positioned at its 5′-end or at 1-5 nucleotides apart from the 5′-end. For example, the fluorescent reporter molecule is positioned at its 5′-end of the labeled primer and the quencher molecule positioned at 5-50 nucleotides apart from the reporter molecule.

According to a preferred embodiment, the fluorescent reporter molecule on the labeled primer is positioned at its 5′-end or at 1-5 nucleotides apart from the 5′-end, most preferably at its 5′-end.

The reporter molecule and the quencher molecule useful in the present invention may include any one known to those of skill in the art. Examples of those are: Cy2™ (506), YO-PRO™-1 (509), YOYO™-1 (509), Calcein (517), FITC (518), FluorX™ (519), Alexa™ (520), Rhodamine 110 (520), 5-FAM (522), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), DiI (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red(615), Nile Red (628), YO-PRO™-3 (631), YOYO™-3 (631), R-phycocyanin (642), C-Phycocyanin (648), TO-PRO™-3 (660), TOTO3 (660), DiD DiIC(5) (665), Cy5™ (670), Thiadicarbocyanine (671) and Cy5.5 (694). The numeric in parenthesis is a maximum emission wavelength in nanometer.

It is noteworthy that a non-fluorescent black quencher molecule capable of quenching a fluorescence of a wide range of wavelengths or a specific wavelength may be used in the present invention.

Suitable pairs of reporter-quencher are disclosed in a variety of publications as follows: Pesce et al., editors, Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2^{nd} Edition (Academic Press, New York, 1971); Griffiths, Color AND Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); Haugland, R. P., Handbook of Fluorescent Probes and Research Chemicals, 6^{th} Edition, Molecular Probes, Eugene, Oreg., 1996; U.S. Pat. Nos. 3,996,345 and 4,351,760.

In the FRET label adapted to the label primer, the reporter encompasses a donor of FRET and the quencher encompasses the other partner (acceptor) of FRET. For example, a fluorescein dye is used as the reporter and a rhodamine dye as the quencher.

At least three primer pairs for the secondary nested real-time multiplex PCR may have any form or structure so long as they comprise a complementary sequence to the target nucleic acid sequence.

In one embodiment, at least one primer of each pair of at least three primer pairs for the secondary nested real-time multiplex PCR has the DPO structure as described above.

The labeled primers useful in the present invention include any labeled primer known in the art for generating signals in conventional real-time PCR processes. They may include additional structural feature or require specific polymerization conditions depending on the type of the labeled primers.

The illustrated examples of the labeled primers include, but not limited to, Plexor™ (Sherrill C B, et al., Journal of the American Chemical Society 126:4550-4556 (2004)), Antiprimer (Jin Li, et al., Clinical Chemistry 52:4 624-633 (2006)), Individual dye-labeled oligonucleotides (U.S. Pat. No. 6,593,091), LUX™ (I. A. Nazarenko, et al. Nucleic Acids Res, 30:2089-2095 (2002); U.S. Pat. No. 7,537,886), TSG primer (KR Appln. No. 10-2009-0127880), THD primer (PCT Appln. No. PCT/KR2009/007064), Lion (U.S. Pat. No. 6,248,526), Self-quenching signal primer (U.S. Pat. Nos. 5,846,726 and 6,054,279), Sunrise (I. A. Nazarenko, et al., Nucleic acids Research, 125(12):2516-2521 (1997); Hernandez M et, al. J. cereal Sci, 39:99-107 (2004), U.S. Pat. Nos. 5,866,336, 6,090,552 and 6,117,635) and Scorpions (David Whitcombe, et al., Nature Biotechnology, 17:804-807 (1999) and EP Pat. No. 1,088,102).

Where Plexor™ (Sherrill C B, et al., Journal of the American Chemical Society 126:4550-4556 (2004)) is used as the labeled primer, it is preferable that Dabcyl-iso-dGTP is employed for quenching.

Where Antiprimer (Jin Li, et al., Clinical Chemistry 52:4 624-633 (2006)) is used as the labeled primer, it is preferable that anti-primer is additionally employed for quenching free primers.

Where Individual dye-labeled oligonucleotides (U.S. Pat. No. 6,593,091) are used as the labeled primer, it is preferable that two primers each having a single label are employed.

Among the illustrated examples of the labeled primers, the THD primer and TSG primer had been developed by the present inventor. In accordance with my findings associated with the primers, either THD primer or TSG primer successfully produces signals for the detection of at least three target nucleic acid sequences in real-time multiplex PCR amplifications. In addition, the present inventor had obtained a surprising discovery that the signals from the THD primer or TSG primer were generated by its conformational change or the cleavage of its 5′-end.

According to a preferred embodiment, the template-dependent nucleic acid polymerase used in the step (c) is a nucleic acid polymerase without the 5′ to 3′ nuclease activity, a nucleic acid polymerase with the 5′ to 3′ nuclease activity, a nucleic acid polymerase with the 3′ to 5′ exonuclease activity or their combination. Preferably, the template-dependent nucleic acid polymerase is a nucleic acid polymerase without the 5′ to 3′ nuclease activity, a nucleic acid polymerase with the 5′ to 3′ nuclease activity or their combination.

In the primer signaling process, the signals to be finally detected are preferably ascribed to incorporation of labeled primers into double-stranded PCR products, conformational change of labeled primers or cleavage of the 5′-end of labeled primers.

For instance, where the labeled primer has a single label, the label provides a signal by the increase of its fluorescence intensity when it is incorporated into the double-stranded PCR product.

Where the labeled primer has an interactive dual label system composed of a reporter molecule and a quencher molecule and is not hybridized with a target nucleic acid sequence, the quencher molecule is spatially adjacent to the reporter molecule such that it quenches signal from the reporter molecule. Where the interactive dual labeled primer is hybridized with a target nucleic acid sequence, the quencher molecule is spatially apart from the reporter molecule such that it no longer quenches signal from the reporter molecule. By the conformational change of the interactive dual labeled primer, signals indicative of the presence of the target nucleic acid sequences can be obtained.

In such a signal generation process, the labeled primer system is responsible for signal generation as well as target amplification, which is different from the labeled probe system.

In the case that signals generated by the incorporation of labeled primers into double-stranded PCR products or by the conformational change of labeled primers are detected for target nucleic acid sequences, the nucleic acid polymerase without the 5′ to 3′ nuclease activity can be utilized.

The exemplified nucleic acid polymerases without the 5′ to 3′ nuclease activity include Klenow fragment of E. coli DNA polymerase I, thermostable DNA polymerase and bacteriophage T7 DNA polymerase.

Alternatively, in accordance with my previous findings, where the labeled primer has an interactive label system composed of a reporter molecule and a quencher molecule, its 3′-end is extended and its 5′-end is cleaved upon contacting to the nucleic acid polymerase with the 5′ to 3′ nuclease activity, followed by the release of either the reporter molecule or the quencher molecule to generate signals indicative of the presence of the target nucleic acid sequences. This labeled primer is called “Target hybridization Detection (THD)” primer. In this signal generation strategy, the THD primer has a dual function involving target amplification and signal generation.

In the case that signals generated by the cleavage of the 5′-end of labeled primers (e.g. THD primer) are detected for the presence of target nucleic acid sequences, the nucleic acid polymerase with the 5′ to 3′ nuclease activity is required.

According to a preferred embodiment, the template-dependent nucleic acid polymerase having the 5′ to 3′ nuclease activity is a thermostable DNA polymerase which may be obtained from a variety of bacterial species, including Thermus aquaticus (Taq), Thermus thermophllus (Tth), Thermus filiformis, Thermis flavus, Thermococcus literalis, Pyrococcus furiosus (Pfu), Thermus antranikianii, Thermus caldophilus, Thermus chliarophllus, Thermus flavus, Thermus igniterrae, Thermus lacteus, Thermus oshimai, Thermus ruber, Thermus rubens, Thermus scotoductus, Thermus silvans, Thermus species Z05, Thermus species sps 17, Thermus thermophilus, Thermotoga maritima, Thermotoga neapolitana and Thermosipho africanus. Most preferably, the template-dependent nucleic acid polymerase having the 5′ to 3′ nuclease activity is Taq DNA polymerase.

When the nucleic acid polymerase with the 3′ to 5′ exonuclease activity is employed, the labeled primer comprises an artificial mismatch nucleotide sequence on its 3′-end portion.

The mismatch nucleotide(s) can be located on various positions of the 3′-end portion. According to a preferred embodiment, the mismatch nucleotide is present at its 3′-end or 1-5 nucleotides apart from its 3′-end, more preferably at its 3′-end or 1-2 nucleotides apart from its 3′-end, still more preferably at its 3′-end or 1 nucleotides apart from its 3′-end, most preferably, at its 3′-end of primers.

The number of the mismatch nucleotides may be 1-5, preferably 1-4, more preferably 1-3, still more preferably 1-2 and most preferably 1. Where primers carry at least 2 mismatch nucleotides, the mismatch nucleotides may be located continuously or intermittently.

According to a preferred embodiment, the fluorescent reporter molecule or the quencher molecule on the labeled primer is positioned at its 3′-end or at 1-5 nucleotides apart from the 3′-end, more preferably at its 3′-end. When the primer is hybridized with the target nucleic acid sequence, its 3′-end having the fluorescent reporter molecule or the quencher molecule is digested with the nucleic acid polymerase with the 3′ to 5′ exonuclease activity to release the fluorescent reporter molecule or the quencher molecule, thereby unquenching fluorescence signal from the reporter molecule to obtain the fluorescence signal indicating the target nucleic acid sequence.

According to a preferred embodiment, the template-dependent nucleic acid polymerase with the 3′ to 5′ exonuclease activity is a thermostable DNA polymerase which may be obtained from a variety of bacterial species, including Thermus filiformis, Thermis flavus, Thermococcus Pyrococcus furiosus (Pfu), Thermus antranikianii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana and Thermosipho africanus. Most preferably, the template-dependent nucleic acid polymerase having the 3′ to 5′ nuclease activity is Pfu DNA polymerase.

Finally, the signals indicative of the presence of the target nucleic acid sequences are detected. The signal detection may be performed for each cycle of the repetition, at the end of the repetition or at each of predetermined time intervals during the repetition. Preferably, the signal detection may be performed for each cycle of the repetition to improve the detection accuracy.

The signal may be detected or measured by conventional methods for each label. For example, the fluorescence signal may be detected or measured by conventional methods, e.g., fluorometers.

In another aspect of this invention, there is provided a kit for a real-time multiplex PCR detection of at least three types of target nucleic acid sequences with elimination of false positive signals, which comprises:

(a) at least three primer pairs used for a primary multiplex PCR of the at least three types of target nucleic acid sequences to obtain the amplicons of the target nucleic acid sequences in the sample; and

(b) at least three primer pairs for a secondary nested real-time multiplex PCR of the amplicons in which at least one primer of each pair of the at least three primer pairs is a nested primer hybridizable with an internal sequence of the corresponding amplicon, wherein each pair of the at least three primer pairs comprises at least one nested primer having a label generating a signal indicative of the presence of the target nucleic acid sequence during the secondary nested real-time multiplex PCR, such that the secondary nested real-time multiplex PCR can be carried out for the number of cycles not to generate the false signals but to generate the signals indicative of the presence of the target nucleic acid sequences.

Since the kit of this invention is constructed to perform the nested multiplex real-time PCR method of the present invention described above, the common descriptions between them are omitted in order to avoid undue redundancy leading to the complexity of this specification.

According to a preferred embodiment, the kit further comprises a template-dependent nucleic acid polymerase.

Preferably, the secondary nested real-time multiplex PCR is carried out for no more than 30 cycles, more preferably 25 cycles, still more preferably no more than 20 cycles.

According to a preferred embodiment, when the primer used in the secondary nested real-time multiplex PCR has the label generating the signal indicative of the presence of the target nucleic acid sequence, the primer is a nested primer hybridizable with an internal sequence of the corresponding amplicon.

The present kits may optionally include the reagents required for performing target amplification PCR reactions (e.g., PCR reactions) such as buffers, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity.

The kits may also include reagents necessary for performing positive and negative control reactions. Optimal amounts of reagents to be used in a given reaction can be readily determined by the skilled artisan having the benefit of the current disclosure. The kits, typically, are adapted to contain in separate packaging or compartments the constituents afore-described.

The features and advantages of the present invention will be summarized as follows:

(a) Elimination of False Positive Signals Generated by the Dimer Formation of Labeled Primers

The conventional real-time multiplex PCR methods require multiple labeled primers being very likely to form dimer structures causing the generation of false positive signals, which is considered serious shortcomings and limitations in the conventional real-time multiplex PCR method. In contrast, by using pre-amplified product, labeled nested primers and a control of cycle numbers, the present invention method fundamentally eliminates false positive signals generated by the dimer formation of the labeled primers.

(b) Elimination of False Positive Signals in a Negative Control

The conventional real-time multiplex PCR methods may have false positive signal problems in a negative control having no template due to the dimer formation of multiple labeled primers. However, the present method prevents such false positive signal problems in the negative control having no template by completely eliminating the false positive signals generated by the dimer formation of the labeled primers.

(c) Elimination of False Positive Signals Generated by Non-Specific Hybridization of Labeled Primers with Non-Target Sequences

According to common knowledge known to one of skill in the art, labeled primers are likely to be non-specifically hybridized with non-target nucleic acid sequences in multiplex target detection such that false positive signals are produced. In the present method, since the secondary nested real-time multiplex PCR uses labeled primers as nested primers, leading to similar effects to conventional nested PCR, the target specificity of the present invention is highly increased to prevent occurrence of false positive signals by non-specific hybridization.

(d) Elimination of False Negative Signals in the Detection of Very Low Copy Number of Target Sequences

Target sequences present in very low amounts may be analyzed to be negative by conventional real-time multiplex PCR methods. However, since the secondary nested real-time multiplex PCR of the present invention is performed using pre-amplified products, the present method enables to detect the very low amount of target sequences and prevent the false negative signals.

(e) As discussed hereinabove, the primer used in the present invention having the DPO structure gives rise to the improvement of binding specificity, thereby eliminating false positive signals associated with non-target binding of the primer in multiplex PCR.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### The Effect of the Nested Real-Time Multiplex PCR Method in Elimination of False Positive Signals Generated by the Dimer Formation of Labeled Primers During Real-Time Multiplex PCR for Chlamydia trachomatis, Neisseria gonorrhoeae and Trichomonas vaginalis

The nested real-time multiplex PCR method of the present invention was examined in comparison with the conventional real-time multiplex PCR method in order to verify its effects on elimination of the false positive signals resulting from the dimer formation of the labeled primers.

For this study, the primer pairs for the primary multiplex PCR and the labeled nested primers for the secondary nested real-time multiplex PCR were designed for the detection of the three different types of target nucleic acids of C. trachomatis, N. gonorrhoeae and T. vaginalis.

The primer pairs for the primary multiplex PCR were designed to have the dual priming oligonucleotide (DPO) structure for enhancement in target specificity.

For the secondary nested real-time multiplex PCR, the dual labeled primers were used as the labeled primers for target signal generation in this Example. The dual labeled primers have a fluorescent reporter molecule such as FAM, Alexa 532 or Alexa 647 at their 5′-end and a quencher molecule such as Black Hole quencher 1 (BHQ-1) or Black Hole quencher 2 (BHQ-2). The fluorescent reporter molecule and the quencher molecule are separated by 18 or 19 nucleotides. The dual labeled primers are nested primers hybridizable with the internal sequences of the corresponding amplicons generated by the primary multiplex PCR. The annealing to targets nucleic acid sequences and extension of the dual labeled nested primers induces generation of fluorescence signals. For the secondary nested real-time multiplex PCR, the dual labeled nested primer was employed together with one primer from the primer pair used for the primary multiplex PCR.

The combination of labeled nested primers having conventional or DPO structure was used in order to examine whether the present method is able to eliminate the false positive signals generated by the dimer formation of the labeled nested primes in such combination.

The same primer pairs were used for both the secondary nested real-time multiplex PCR and the conventional real-time multiplex PCR.

Only one type of template (genomic DNA of N. gonorrhoeae) was used to elucidate the features of the present method accomplishing elimination of the false positive signals.

The primer sequences used in this Example are:

Primer set for C. trachomatis
CT_F:
(SEQ ID NO: 1)
5′-TGCAACGGGTTATTCACTCCCIIIIICATTGAAACT-3
CT_R:
(SEQ ID NO: 2)
5′-ACCCATACCACACCGCTTTCTIIIIIGCCTACACGT-3′
CT_DLP(18):
(SEQ ID NO: 3)
5′-[AminoC6 + Alexa532] CACCTTCTCGTACCAAAGC
[BHQ1-dT]AGAA-3′
Primer set for N. gonorrhoeae
NG_F:
(SEQ ID NO: 4)
5′-CTATTTTTATGAGCCGGAACCGIIIIIAAGCGTGGGA-3′
NG_R:
(SEQ ID NO: 5)
5′-TTGAAGTTGTCGGGAAAGCCIIIIITTCTTTGCAAC-3′
NG_DLP(18):
(SEQ ID NO: 6)
5′-[6-FAM]CCCCGTGCTTTTCCATCTT[BHQ1-dT]IIIIITTG
CGGGT-3′
Primer set for T. vaginalis.
TV_F:
(SEQ ID NO: 7)
5′-CCCAAAGGCTAACCGTGAGATTTTTATCTCCCTCA-3′
TV_R:
(SEQ ID NO: 8)
5′-TCGGCTGTTGTGTTGAAAGCTTTTTCACGCTCT-3′
TV_DLP(19):
(SEQ ID NO: 9)
5′-[AminoC6 + Alexa647]TGATCTCACAGCCTGGATGG
[BHQ2-dT]CA-3′
(I: Deoxyinosine)

### Conventional Real-Time Multiplex PCR Method

The conventional real-time multiplex PCR was conducted with 1000 fg of genomic DNA of N. gonorrhoeae as a template or without the template in the final volume of 20 μl containing, 10 μl of 2× QuantiTect Multiplex PCR Master Mix (Qiagen) [11 mM of MgCl₂, QuantiTect Multiplex PCR buffer, HotstarTaq DNA polymerase and dNTP mix], and 5 pmole of each of primers (SEQ ID NOs: 1, 3, 4, 6, 8 and 9); the tubes containing the reaction mixture were placed on the real-time thermocycler (CFX96, Bio-Rad); the reaction mixture was denatured for 15 min at 95° C. and subjected to 40 cycles of 30 sec at 94° C., 60 sec at 55° C. and 10 sec at 72° C. Detection of the generated signal was performed at the annealing step (55° C.) of each cycle.

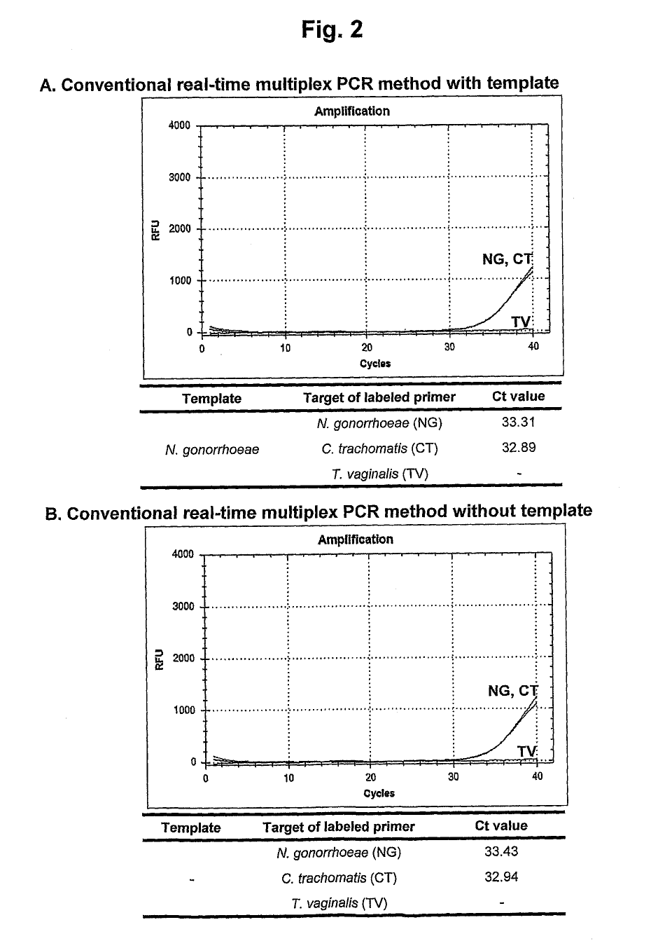

As shown in panel A of FIG. 2, the conventional real-time multiplex PCR method generated signal of C trachomatis (CT) (Ct value: 32.89) as well as signal of N. gonorrhoeae (NG) (Ct value: 33.31) although only genomic DNA of N. gonorrhoeae was used as a template, indicating that the signal of CT is false positive.

The conventional real-time multiplex PCR without any template induced signal generation from both NG (Ct value: 33.43) and CT (Ct value: 32.94) as shown in panel B of FIG. 2. These results indicate that the false positive signals are generated by the dimer formation of labeled primers. Therefore, the false positive signal (CT) of panel A is demonstrated to be generated by the dimer formation of labeled primers.

Furthermore, the signal of NG in panel A turned out to be true but the signal of NG in panel B was false positive although the signals in panels A and B were observed to be produced with a similar pattern. These results address that true signals and false signals may not be discriminated in the conventional real-time multiplex PCR method.

It could be appreciated that the conventional real-time multiplex PCR methods for the detection of target sequences are very likely to accompany false positive signals owing to the dimer formation of labeled primers, making it difficult to obtain false-free detection results for target sequences.

### Nested Real-Time Multiplex PCR Method of the Present Invention

### Primary Multiplex PCR

The primary multiplex PCR was conducted with 1000 fg of genomic DNA of N. gonorrhoeae as a template or without the template in the final volume of 20 μl containing 10 μl of 2× Multiplex Master Mix (Qiagen) [6 mM of MgCl₂, HotstarTaq PCR buffer, HotstarTaq DNA polymerase and dNTP mix], and 5 pmole of each of primers (SEQ ID NOs: 1, 2, 4, 5, 7 and 8); the tubes containing the reaction mixture were placed on the thermocycler (ABI9700, Applied BioSystems); the reaction mixture was denatured for 15 min at 95° C. and subjected to 35 cycles of 30 sec at 94° C., 60 sec at 55° C. and 60 sec at 72° C.

### Secondary Nested Real-Time Multiplex PCR

The secondary nested real-time multiplex PCR was conducted in the final volume of 20 μl containing 1 μl of the primary multiplex PCR product, 10 μl of 2× QuantiTect Multiplex PCR Master Mix (Qiagen) [11 mM of MgCl₂, QuantiTect Multiplex PCR buffer, HotstarTaq DNA polymerase and dNTP mix] and 5 pmole of each of primers (SEQ ID NOs: 1, 3, 4, 6, 8 and 9); the tubes containing the reaction mixture were placed on the real-time thermocycler (CFX96, Bio-Rad); the reaction mixture was denatured for 15 min at 95° C. and subjected to 30 cycles of 30 sec at 94° C., 60 sec at 55° C. and 10 sec at 72° C. Detection of the generated signal was performed at the annealing step (55° C.) of each cycle.

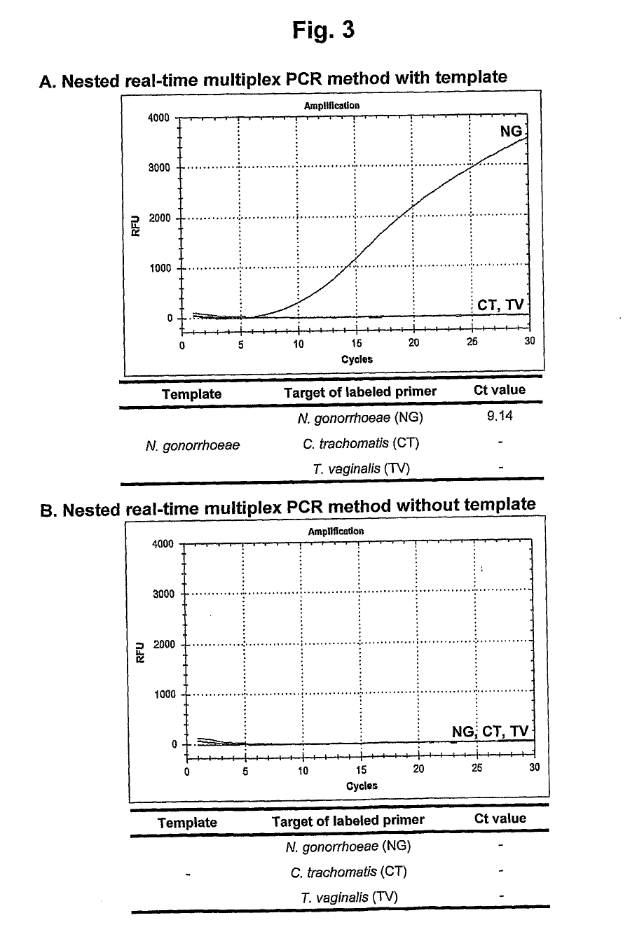

As represented in panel A of FIG. 3, when only N. gonorrhoeae (NG) was used a template, the present method showed only target signal of N. gonorrhoeae (NG) (Ct value: 9.14) without any false positive signals. In addition, in the negative control having no template, the present method did not show any false positive signals as shown in panel B of FIG. 3.

These results demonstrate that the present method permits to accurately detect target nucleic acid sequences with elimination of false positive signals generated by the dimer formation of labeled primers in a real-time multiplex PCR manner.

### Example 2

### The Effect of the Nested Real-Time Multiplex PCR Method in Elimination of False Positive Signals Generated by the Dimer Formation of Labeled Primers During Real-Time Multiplex PCR for Haemophilus ducreyi, Herpes Simplex Virus 1 and Herpes Simplex Virus 2

As reproducibility experiments of the present invention, three different target nucleic acid sequences of H. ducreyi, Herpes simplex virus 1 and Herpes simplex virus 2 were used. The design of the primers and the conditions of the experiment were the same manner as the Example 1, except of primer sequences and labeling. The dual labeled primers have at their 5′-end a fluorescent reporter molecule such as Alexa 532, Alexa 594 or Alexa 647 and a quencher molecule such as Black Hole quencher 1 (BHQ-1) or Black Hole quencher 2 (BHQ-2). The fluorescent reporter molecule and the quencher molecule are separated by 18, 20 or 21 nucleotides. As a template, 1000 fg of the genomic DNA of Herpes simplex virus 1 was used.

The same primer pairs were used for both the secondary nested real-time multiplex PCR and the conventional real-time multiplex PCR,

The primer sequences used in this Example are:

Primer set for H. ducreyl,
HD_F:
(SEQ ID NO: 10)
5′-AAAGAACGTGAAAAAGCCGACCIIIIIAAAATTACTA-3′
HD_R:
(SEQ ID NO: 11)
5′-ATAGCCCAGAAGGGTTAGCAATIIIIIGACAATCAAT-3′
HD_DLP(20):
(SEQ ID NO: 12)
5′-[AminoC6 + Alexa532]CCTCGGCTGGTATTACGACTA
[BHQ1-dT]CIIIIIAGCCTAGG-3′
Primer set for Herpes simplex virus 1
HSV1_F:
(SEQ ID NO: 13)
5′-GTCCTGGTGGTGCAACCGIIIIIAGTTCCGA-3′
HSV1_R:
(SEQ ID NO: 14)
5′-ATACGCACGGTCACCCCCACIIIIITGTGAGACT-3′
HSV1_DLP(18):
(SEQ ID NO: 15)
5′-[AminoC6 + Alexa594]CAGCCGATTACGACGAGGA
[BHQ2-dT] IIIIITGACGAGG-3′
Primer set for Herpes simplex virus 2
HSV2_F:
(SEQ ID NO: 16)
5′-GTCGTCTGCGCCAAATACGIIIIIGCAGACCC-3′
HSV2_R:
(SEQ ID NO: 17)
5′-CAGGCGATGGTCAGGTTGTIIIIITGCTTTCG-3′
HSV2_DLP(21):
(SEQ ID NO: 18)
5′-[AminoC6 + Alexa647]CCGATCACTGTGTACTACGCAG
[BHQ2-dT]IIIIIAACGTGCC-3′
(I: Deoxyinosine)

### Conventional Real-Time Multiplex PCR Method

The conventional real-time multiplex PCR was conducted as the Example 1, except for primers (SEQ ID NOs: 10, 12, 14, 15, 17 and 18) and template (Herpes simplex virus 1).

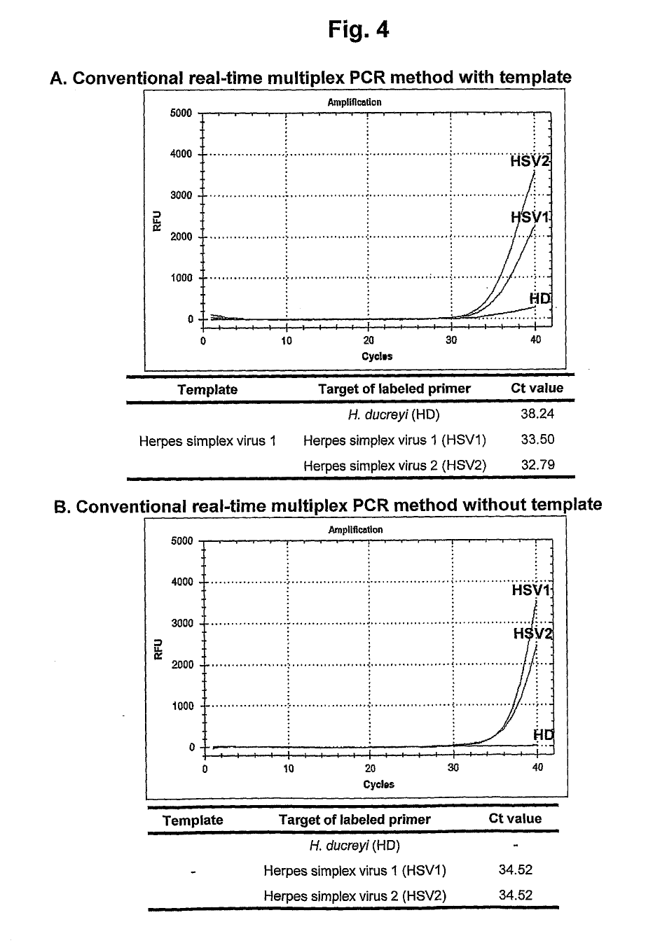

As shown in panel A of FIG. 4, the conventional real-time multiplex PCR method generated signals of H. ducreyi (HD) (Ct value: 38.24) and Herpes simplex virus 2 (HSV2) (Ct value: 32.79) as well as signal of Herpes simplex virus 1 (HSV1) (Ct value: 33.50) although only genomic DNA of HSV1 was used as a template, indicating that the signals of HD and HSV2 are false positive.

The conventional real-time multiplex PCR without any template induced signal generation from both HSV1 (Ct value: 34.52) and HSV2 (Ct value: 34.52) as shown in panel B of FIG. 4. These results indicate that the false positive signals (HSV1 and HSV2) are generated by the dimer formation of labeled primers.

Therefore, the false positive signal of HSV2 in panel A is demonstrated to be generated by the dimer formation of labeled primers and the false positive signal of HD in panel A is indicated to be generated by non-specific hybridization of labeled primers.

Furthermore, the signals of HSV1 and HSV2 in panels A and B were generated regardless of the presence of target templates. These results address that true signals and false signals can not be discriminated in the conventional real-time multiplex PCR method.

### Nested Real-Time Multiplex PCR Method of the Present Invention

### Primary Multiplex PCR

The primary multiplex PCR was conducted as the Example 1, except for primers (SEQ ID NOs: 10, 11, 13, 14, 16 and 17) and template (Herpes simplex virus 1).

### Secondary Nested Real-Time Multiplex PCR

The secondary nested real-time multiplex PCR was conducted as the Example 1, except for primers (SEQ ID NOs: 10, 12, 14, 15, 17 and 18).

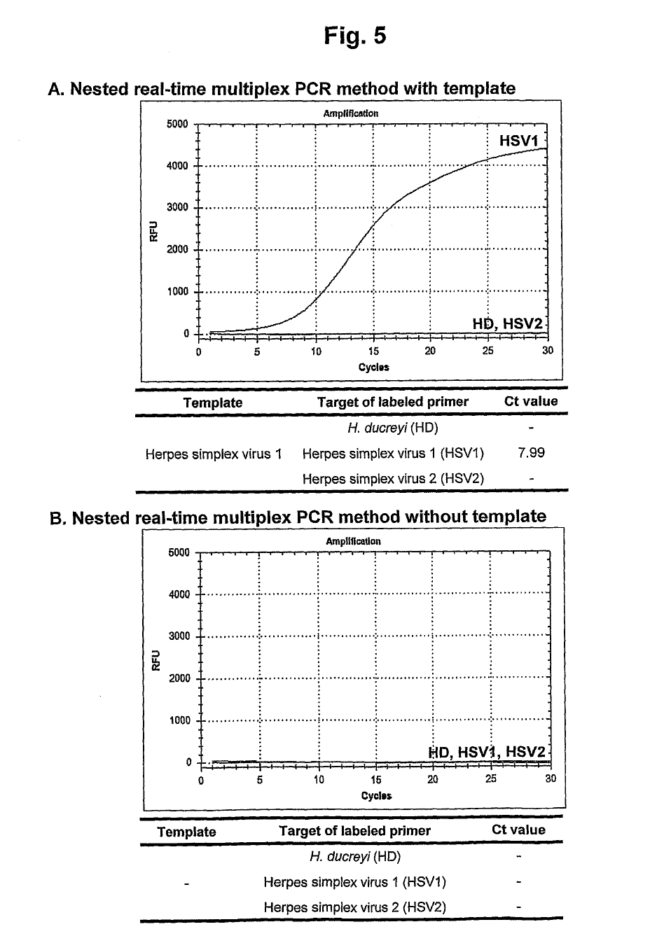

As represented in panel A of FIG. 5, when only Herpes simplex virus 1 (HSV1) was used a template, the present method showed only target signal of Herpes simplex virus 1 (HSV1) (Ct value: 7.99) without any false positive signals. In addition, in the negative control having no template, the present method did not show any false positive signals as shown in panel B of FIG. 5.

These results demonstrate that the present method permits to accurately detect target nucleic acid sequences with elimination of false positive signals generated by the dimer formation of labeled primers in a real-time multiplex PCR manner.

### Example 3

### Sensitivity and Specificity in the Nested Real-Time Multiplex PCR Method of the Present Invention

The sensitivity and specificity of the nested real-time multiplex PCR method of the present invention were tested by detecting the genomic DNA of H. ducreyi or Herpes simplex virus 1 in the presence of three primer pairs for the detection of H. ducreyi, Herpes simplex virus 1 and Herpes simplex virus 2.

The same primer pairs described in Example 2 were used and the serially diluted genomic DNA of H. ducreyi or Herpes simplex virus 1 was used as a template.

### Primary Multiplex PCR

The primary multiplex PCR was conducted in the final volume of 20 μl containing the serially diluted genomic DNA of H. ducreyi or Herpes simplex virus 1 (1000 fg, 100 fg, 10 fg or 1 fg), 10 μl of 2× Multiplex Master Mix (Qiagen) [6 mM of MgCl₂, HotstarTaq PCR buffer, HotstarTaq DNA polymerase and dNTP mix], 5 pmole of each of primers (SEQ ID NOs: 10, 11, 13, 14, 16 and 17); the tube containing the reaction mixture was placed on the thermocycler (ABI9700, Applied BioSystems); the reaction mixture was denatured for 15 min at 95° C. and subjected to 35 cycles of 30 sec at 94° C., 60 sec at 55° C. and 60 sec at 72° C.

### Secondary Nested Real-Time Multiplex PCR

The secondary nested real-time multiplex PCR of the present method was conducted in the final volume of 20 μl containing 1 μl of the primary multiplex PCR product, 10 μl of 2× QuantiTect Multiplex PCR Master Mix (Qiagen) [11 mM of MgCl₂, QuantiTect Multiplex PCR buffer, HotstarTaq DNA polymerase and dNTP mix], 5 pmole of each of primers (SEQ ID NOs: 10, 12, 14, 15, 17 and 18); the tube containing the reaction mixture was placed on the real-time thermocycler (CFX96, Bio-Rad); the reaction mixture was denatured for 15 min at 95° C. and subjected to 30 cycles of 30 sec at 94° C., 60 sec at 55° C. and 10 sec at 72° C. Detection of the generated signal was performed at the annealing step (55° C.) of each cycle.

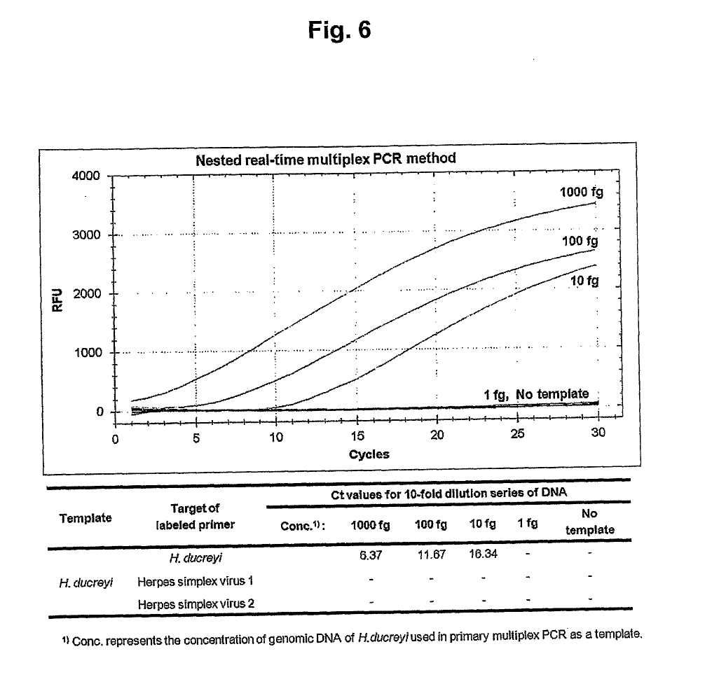

The specificity and sensitivity of the present method were examined to successfully detect the target nucleic acid sequence of H. ducreyi, as shown in FIG. 6.

Although three different labeled primers were used in the secondary real-time multiplex PCR reaction mixture for the simultaneous detection of H. ducreyi (HD), Herpes simplex virus 1 (HSV1) and Herpes simplex virus 2 (HSV2), only the target signal of HD was detected without any signals of HSV1 and HSV2, showing the target specificity of the present method.

Furthermore, the present method could detect up to 10 fg of the genomic DNA of H. ducreyi, as shown in FIG. 6.

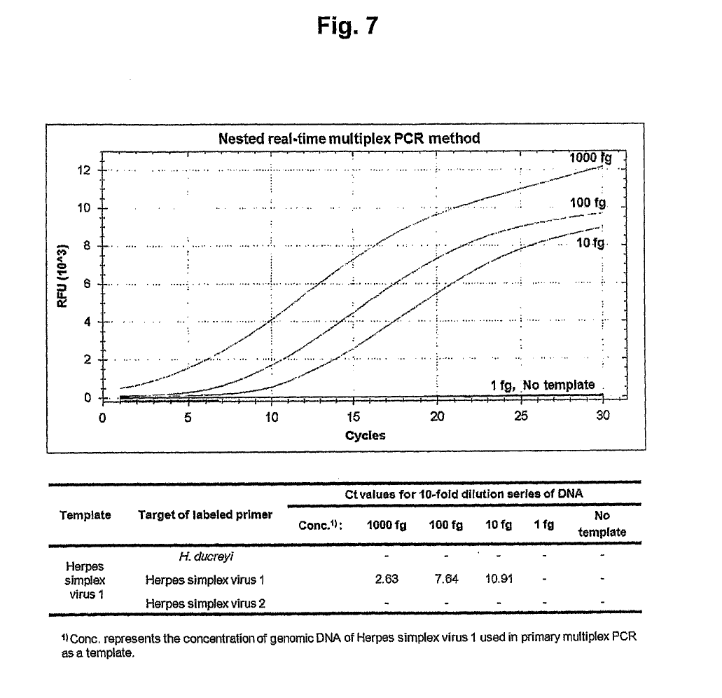

FIG. 7 showed another example of the specificity and sensitivity of the present method by detecting the target nucleic acid sequence of Herpes simplex virus 1.

Although three different labeled primers were used in the secondary real-time multiplex PCR reaction mixture for the simultaneous detection of H, ducreyi (HD), Herpes simplex virus 1 (HSV1) and Herpes simplex virus 2 (HSV2), only the target signal of HSV1 was detected without any signals of HD and HSV2, showing the target specificity of the present method.

Furthermore, the another example also showed that the present method detected up to 10 fg of the genomic DNA of Herpes simplex virus 1, as shown in FIG. 7.

Taken together, it could be understood that the present method enables to detect target sequences in a real-time multiplex manner with enhanced sensitivity and specificity.

### Example 4

### Multiple Target Detection Using the Nested Real-Time Multiplex PCR Method of the Present Invention

To examine whether at least three different target nucleic acid sequences could be simultaneously detected in a real-time manner by the present method, the mixed genomic DNA samples of H. ducreyi, Herpes simplex virus 1 and Herpes simplex virus 2 were used as templates and the same primer pairs in Example 2 were used.

### Primary Multiplex PCR

The primary multiplex PCR was conducted as the Example 2, except for template (1000 fg of each genomic DNA of H. ducreyi, Herpes simplex virus 1 and Herpes simplex virus 2)

### Secondary Nested Real-Time Multiplex PCR

The secondary nested real-time multiplex PCR was conducted as the Example 2.

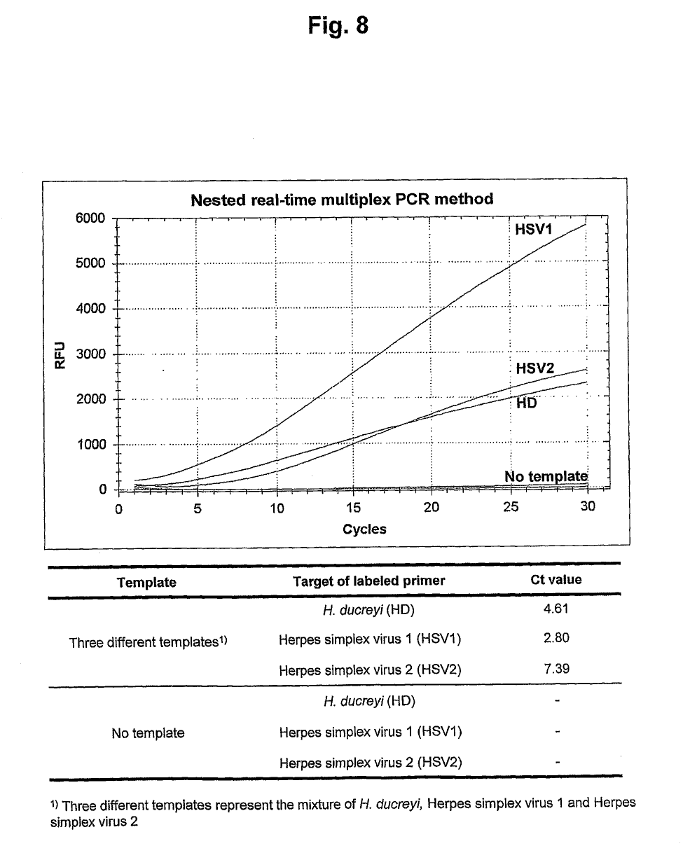

As shown in FIG. 8, the nested real-time multiplex PCR method of present invention simultaneously detected three different target signals for the presence of H. ducreyi (HD) (Ct; 4.61), Herpes simplex virus 1 (HSV1) (Ct; 2.80) and Herpes simplex virus 2 (HSV2) (Ct; 7.39). In the negative control having no template, there was no false positive signal.

Consequently, it could be appreciated that the present invention permits to simultaneously detect at least three target nucleic acid sequences with elimination of false positive signals in a real-time multiplex manner.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method of a real-time multiplexing detection of at least three types of target nucleic acid sequences in a sample with elimination of false signals in a real-time multiplex PCR (polymerase chain reaction), which comprises the steps of:
(a) performing a primary multiplex PCR for amplifying the target nucleic acid sequences in a reaction vessel, wherein the primary multiplex PCR is carried out using at least three primer pairs and a template-dependent nucleic acid polymerase under conditions sufficient to amplify the at least three types of target nucleic acid sequences in order to obtain the amplicons of the target nucleic acid sequences in the sample;
(b) preparing a secondary nested real-time multiplex PCR reaction mixture in a reaction vessel distinctly different from the reaction vessel used in the step (a), wherein the secondary nested real-time multiplex PCR reaction mixture contains (i) the amplicons obtained in the step (a), (ii) at least three primer pairs for secondary the nested real-time multiplex PCR of the amplicons in which at least one primer of each pair of the at least three primer pairs is a nested primer hybridizable with an internal sequence of the corresponding amplicon and (iii) a template-dependent nucleic acid polymerase, wherein each pair of the at least three primer pairs comprises at least one nested primer having a label generating the signal indicative of the presence of the target nucleic acid sequence during the secondary nested real-time multiplex PCR;
(c) performing the secondary nested real-time multiplex PCR using the reaction mixture of the step (b) by carrying out at least two cycles of primer annealing, primer extension and denaturation, wherein the signal indicative of the presence of the target nucleic acid sequence is generated from each of the labeled primers during the cycles, such that the secondary nested real-time multiplex PCR can be carried out for the number of cycles not to generate the false signals but to generate the signals indicative of the target nucleic acid sequences; and
(d) detecting the signal indicative of the presence of the target nucleic acid sequence, wherein the detection is performed for each cycle of the repetition of step (c), at the end of the repetition of step (c) or at each of predetermined time intervals during the repetition of step (c), whereby the signals indicative of the target nucleic acid sequences are obtained in a real-time manner without the false signals.

2. The method according to claim 1, wherein the secondary nested real-time multiplex PCR is carried out for no more than 30 cycles.

3. The method according to claim 1, wherein the secondary nested real-time multiplex PCR is carried out for no more than 25 cycles.

4. The method according to claim 1, wherein the secondary nested real-time multiplex PCR is carried out for no more than 20 cycles.

5. The method according to claim 1, wherein when the primer used in the secondary nested real-time multiplex PCR has the label, the primer is a nested primer hybridizable with an internal sequence of the corresponding amplicon.

6. The method according to claim 1, wherein the labels on the at least three primer pairs are different from each other or the same.

7. The method according to claim 6, wherein the label of each pair of at least three primer pairs is different from each other or the same.

8. The method according to claim 1, wherein the labeled primer comprises a single label or an interactive dual label.

9. The method according to claim 8, wherein the label is located on a complementary sequence of the primer to the target nucleic acid sequence.

10-12. (canceled)

13. The method according to claim 1, wherein the template-dependent nucleic acid polymerase in step (b) is a nucleic acid polymerase having no 5′ to 3′ nuclease activity, a nucleic acid polymerase having a 5′ to 3′ nuclease activity, a nucleic acid polymerase having a 3′ to 5′ exonuclease activity or a nucleic acid polymerase having both a 5′ to 3′ nuclease activity and a 3′ to 5′ exonuclease activity.

14. A kit for a real-time multiplex PCR (polymerase chain reaction) detection of at least three types of target nucleic acid sequences in a sample with elimination of false positive signals, which comprises:
(a) at least three primer pairs used for a primary multiplex PCR of the at least three types of target nucleic acid sequences to obtain the amplicons of the target nucleic acid sequences in the sample; and
(b) at least three primer pairs for a secondary nested real-time multiplex PCR of the amplicons in which at least one primer of each pair of the at least three primer pairs is a nested primer hybridizable with an internal sequence of the corresponding amplicon, wherein each pair of the at least three primer pairs comprises at least one nested primer having a label generating a signal indicative of the presence of the target nucleic acid sequence during the secondary nested real-time multiplex PCR, such that the secondary nested real-time multiplex PCR can be carried out for the number of cycles not to generate the false signals but to generate the signals indicative of the presence of the target nucleic acid sequences.

15. The kit according to claim 14, wherein the kit further comprises a template-dependent nucleic acid polymerase.

16. The kit according to claim 14, wherein the secondary nested real-time multiplex PCR is carried out for no more than 30 cycles.

17. The kit according to claim 14, wherein the secondary nested real-time multiplex PCR is carried out for no more than 25 cycles.

18. The kit according to claim 14, wherein the secondary nested real-time multiplex PCR is carried out for no more than 20 cycles.

19. The kit according to claim 14, wherein when the primer used in the secondary nested real-time multiplex PCR has the label, the primer is a nested primer hybridizable with an internal sequence of the corresponding amplicon.

20-21. (canceled)

22. The kit according to claim 14, wherein the labeled primer comprises a single label or an interactive dual label.

23. The kit according to claim 22, wherein the label is located on a complementary sequence of the primer to the target nucleic acid sequence.

24-26. (canceled)

27. The kit according to claim 15, wherein the template-dependent nucleic acid polymerase is a nucleic acid polymerase having no 5′ to 3′ nuclease activity, a nucleic acid polymerase having a 5′ to 3′ nuclease activity, a nucleic acid polymerase having a 3′ to 5′ exonuclease activity or a nucleic acid polymerase having both a 5′ to 3′ nuclease activity and a 3′ to 5′ exonuclease activity.
